(19) United States
(12) Patent Application Publication
(10) Pub. No.: US 2022/0288364 A1
(43) Pub. Date: Sep. 15, 2022

(54) GRADED LINEAR ACCESS DILATOR AND METHODS OF USE

(71) Applicant: Sanford Altman, Miami, FL (US)

(72) Inventor: Sanford Altman, Miami, FL (US)

(21) Appl. No.: 17/198,776

(22) Filed: Mar. 11, 2021

(51) Int. Cl. A61M 29/00

(52) U.S. Cl. CPC A61M 29/00; A61B 2017/3433

(57) ABSTRACT

A tapered dilator includes a plurality of cylindrical segments having a number of different diameters, a plurality of tapered portions, each of the plurality of tapered portions being disposed between adjacent ones of the plurality of cylindrical segments and serving as a transition between the adjacent ones of the plurality of cylindrical segments, and a plurality of markings disposed on an outer surface of each of the plurality of cylindrical segments.

### FIELD OF THE DISCLOSURE

The present disclosure relates to dilators used in medical procedures. More particularly the present disclosure relates to multilevel graduated dilators.

### BACKGROUND OF THE DISCLOSURE

Dilators, as they relate to surgical procedures, are used to enlarge structures, tissues, organs, and/or pathways to gain access to spaces within a patient's body. Dilators work by enlarging the pathway from the region of origin of the dilator, to its intended destination, and are typically used to created tracts or pathways to accommodate medical devices or instruments of specific sizes. Whether used in vascular or non-vascular regions, dilators are often introduced over a wire, positioned in its desired anatomic location prior to advancement of the dilator, allowing the dilator to be safely advanced from its origin to its destination. Current dilators are typically manufactured in individual sizes with the dilator having a tapered tip, starting from its proximal end, to a desired constant diameter throughout the remaining length of the dilator. Often, multiple dilators are required to serially dilate a tract to a specific size, or to achieve a needed result if the original anticipated size was miscalculated. As such, most interventional and/or surgical suites maintain an array of dilators in their inventory.

Despite advances that have been made in this area, there are a variety of problems associated with currently available dilators. For example, many indwelling vascular catheters come with an array of dilators in their implantment kits, allowing the tract to be serially dilated to its desired size. This requires exchange of one dilator over the wire for a second or sometimes third dilator. This is significant as each dilator exchange has associated risks, such as loss of access with the wire accidentally being pulled out of its desired location during the exchange, to bleeding from the vascular tract with exchange from one dilator to the next, to an increase infection risk from these multiple dilator exchanges as compared to one. Thus, multiple dilator exchanges place increased risk on the patient. Additionally, for some vascular procedures a “sheathless” percutaneous approach is preferred, reducing the size of the opening of the vascular structure and resultant damage to the vessel wall created with larger diameter dilatation required by vascular sheaths. For these “sheathless” percutaneous procedures, although the device is of a specific size (e.g., 7 French), a slightly larger dilator may be required (e.g., 7.5 French) to allow for atraumatic percutaneous advancement of the device through the percutaneous tract.

Multiple dilator exchanges unnecessarily extend the operation time, increases the risk for infection and increases healthcare cost for the patient and in the overall system.

Hence, there is a continuing need for improved dilators to address at least some of these concerns.

### SUMMARY OF THE DISCLOSURE

In some examples, a tapered dilator includes a plurality of cylindrical segments having a number of different diameters, a plurality of tapered portions, each of the plurality of tapered portions being disposed between adjacent ones of the plurality of cylindrical segments and serving as a transition between the adjacent ones of the plurality of cylindrical segments, and a plurality of markings disposed on an outer surface of the plurality of cylindrical segments.

In some examples, a tapered dilator includes a plurality of cylindrical segments having a number of different diameters, each of the plurality of cylindrical segments having a distinct color, a plurality of tapered portions, each of the plurality of tapered portions being disposed between adjacent ones of the plurality of cylindrical segments and serving as a transition between the adjacent ones of the plurality of cylindrical segments, and wherein the plurality of cylindrical segments and the plurality of tapered portions define a continuous guidewire lumen extending from a proximal end to a distal end.

### BRIEF DESCRIPTION OF THE DISCLOSURE

Various embodiments of the presently disclosed dilators are disclosed herein with reference to the drawings, wherein:

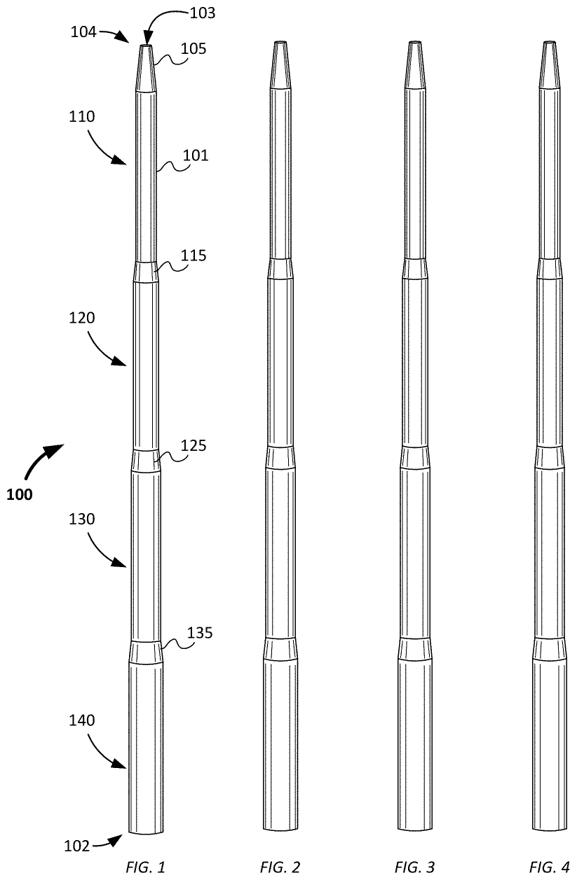

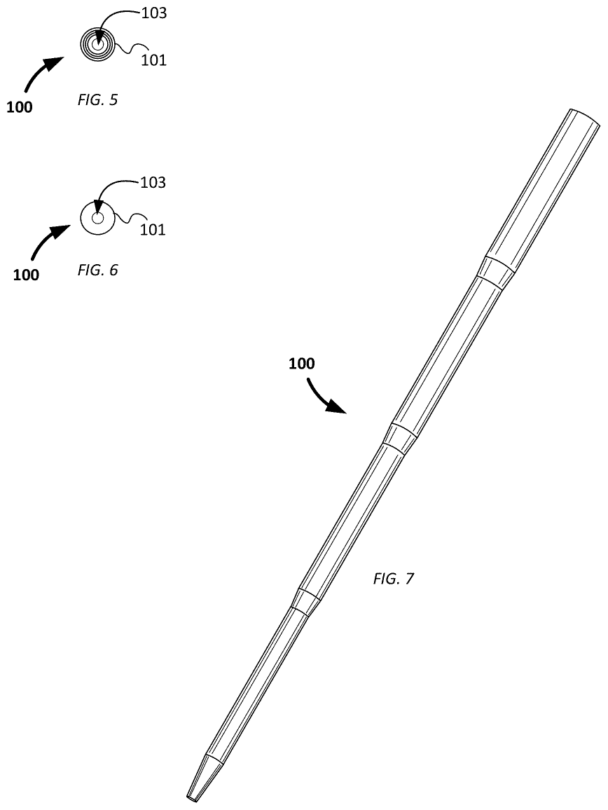

FIGS. 1-7 are schematic front, back, left side, right side, top, bottom and perspective views of a dilator, respectively;

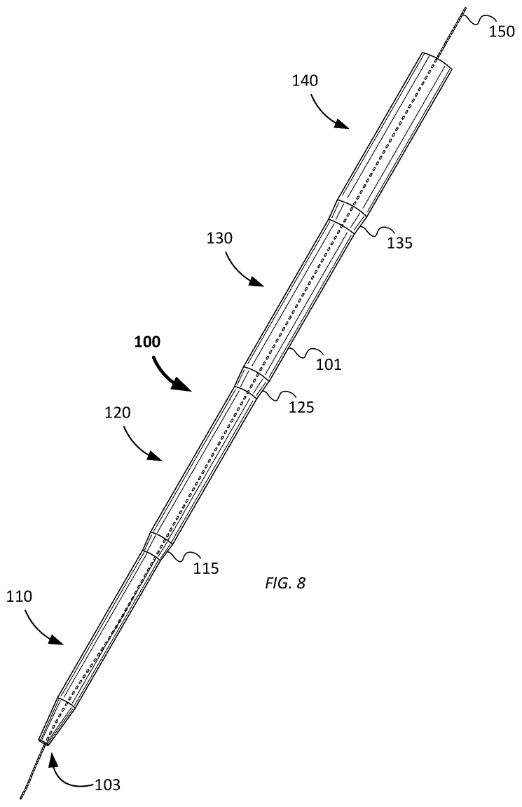

FIG. 8 is a schematic illustration of a dilator being used with a guidewire;

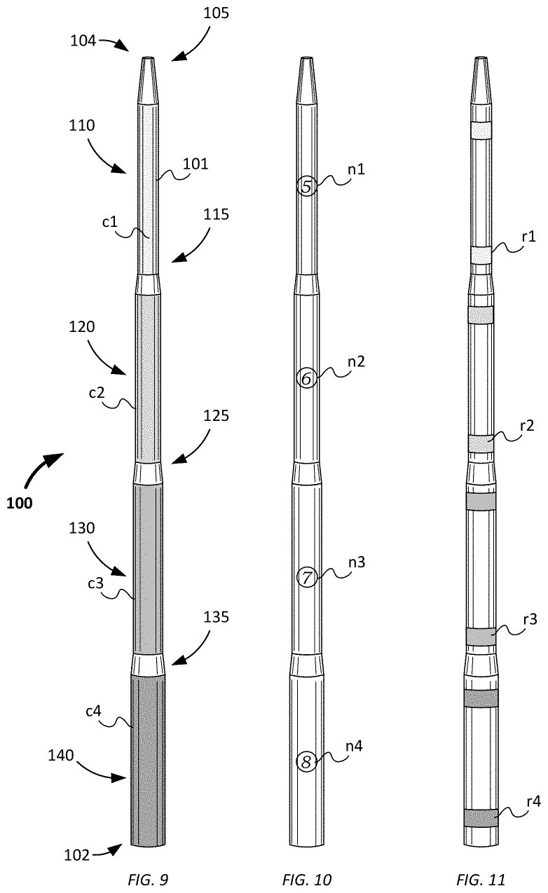

FIG. 9 is a schematic illustration of a dilator with colored segments;

FIG. 10 is a schematic illustration of a dilator with numerical markings; and

FIG. 11 is a schematic illustration of a dilator with annular markings.

Various embodiments of the present. invention will now be described with reference to the appended drawings. It is to be appreciated that these drawings depict only some embodiments which may or may not all be required for functionality of the invention and are therefore not to be considered limiting of its scope.

### DETAILED DESCRIPTION

Despite the various improvements that have been made to dilators, conventional devices suffer from some shortcomings as described. above.

There therefore is a need for further improvements to the devices, systems, and methods of dilating pathways often required in medical and/or surgical care. Among other advantages, the present disclosure may address one or more of these critical needs.

As used herein, the term “proximal,” when used in connection with a component of a dilator, refers to the end of the component closest to the physician, the patient and others when the dilator is inserted in a patient, whereas the term “distal,” when used in connection with a component of a dilator, refers to the end of the component farthest from the physician residing or intended to reside within the desired location. Likewise, the terms “trailing” and “leading” are to be taken as relative to the operator (e.g., physician) of the dilator. “Trailing” is to be understood as relatively close to the operator, and “leading” is to be understood as relatively farther away from the operator.

As shown in FIGS. 1-7, a graded linear access dilator 100 includes a body 101 extending between proximal end 102 and distal end 104. Body 101 may be symmetric about any of its diameters and may be formed of various materials including polymers (e.g., polyethylene terepthalate, polyethylene, high density polyethylene, polypropylene, polytetrafluoroethylene, expanded polytetrafluoroethylene, poly(ethylene-co-vinyl acetate), poly(butyl methacrylate), and co-polymers thereof) or other suitable materials known in the art. Body 101 may include a conical tip 105 at its distal end. The length and gradation of conical tip 105 may vary based on the intended use.

Body 101 may include a plurality of cylindrical segments 110, 120, 130, 140, and a plurality of tapered portions 115, 125, 135 alternating with the plurality of cylindrical segments and being disposed between adjacent ones of the plurality of cylindrical segments to serve as transitions.

Specifically, each of cylindrical segments 110, 120, 130, 140 may have a constant diameter, the diameter corresponding to a French size on the French scale, commonly used to measure the size of an instrument or catheter. Table 1 below provides a comparison of a French Size, a circumference in millimeters and an outer diameter in both millimeters and inches.

| | | |
|---|---|---|
| French | Circumference | Outer diameter |

| Gauge | (mm) | (mm) | (inches) |
|---|---|---|---|
| 3 | 3.14 | 1 | 0.039 |
| 4 | 4.19 | 1.333 | 0.053 |
| 5 | 5.24 | 1.667 | 0.066 |
| 6 | 6.28 | 2 | 0.079 |
| 7 | 7.33 | 2.333 | 0.092 |
| 8 | 8.38 | 2.667 | 0.105 |
| 9 | 9.42 | 3 | 0.118 |
| 10 | 10.47 | 3.333 | 0.131 |
| 11 | 11.52 | 3.667 | 0.144 |
| 12 | 12.57 | 4 | 0.158 |
| 13 | 13.61 | 4.333 | 0.170 |
| 14 | 14.66 | 4.667 | 0.184 |
| 15 | 15.71 | 5 | 0.197 |
| 16 | 16.76 | 5.333 | 0.210 |
| 17 | 17.81 | 5.667 | 0.223 |
| 18 | 18.85 | 6 | 0.236 |
| 19 | 19.90 | 6.333 | 0.249 |
| 20 | 20.94 | 6.667 | 0.263 |
| 22 | 23.04 | 7.333 | 0.288 |
| 24 | 25.13 | 8 | 0.315 |
| 26 | 27.23 | 8.667 | 0.341 |
| 28 | 29.32 | 9.333 | 0.367 |
| 30 | 31.42 | 10 | 0.393 |
| 32 | 33.51 | 10.667 | 0.419 |
| 34 | 35.60 | 11.333 | 0.445 |

As shown, dilator 100 includes four cylindrical segments 110, 120, 130, 140 although it will be understood that a dilator may be formed with two, three, four, five or more cylindrical segments. In some examples, first segment 110 may correspond to a first French size, and successive segments 120, 130, 140 may increase in size by one French size or more. For example, first segment 110 may be 5 French, second segment 120 may be 6 French, third segment 130 may be 7 French, and fourth segment 140 may be 8 French. It will be understood, however, that the dilator may begin at any French size and its diameter may be gradually enlarged to any second French size, and that the difference between adjacent cylindrical segments may be greater than one French (e.g., adjacent cylindrical segments may be 2 French sizes apart in size). Additionally, the lengths of the cylindrical segments may be modified as desired. Although, dilator 100 shows cylindrical segments of the same, or substantially similar lengths, it will be understood that the cylindrical segments may be of different, increasing, or decreasing lengths from proximal end to distal end, or vice versa.

A number of tapered portions 115, 125, 135 alternate with the plurality of cylindrical segments, and serve as smooth transitions. Each of the tapered portions 115, 125, 135 may be conical and include a first diameter adjacent a first end and a second diameter adjacent a second end, the first diameter and the second diameter having a difference of one French. For example, tapered portion 115 may transition the dilator body from a 5 French first segment 110 to a 6 French second segment 120. The lengths of the tapered portions may be modified as desired. Although, dilator 100 shows tapered portions of the same, or substantially similar lengths, it will be understood that the tapered portions may be of different, increasing, or decreasing lengths from proximal end to distal end, or vice versa.

The cylindrical segments and the tapered portions may be fixed relative to one another (e.g., the cylindrical segments and tapered portions do not move or telescope relative to one another). Additionally, the cylindrical segments and tapered portions may form a unitary continuous outer wall.

A continuous guidewire lumen 103 may extend from proximal end 102 to distal end 104, and defined through each of the plurality of cylindrical segments and each of the plurality of tapered portions. In at least some examples, guidewire lumen 103 has a continuous diameter along the length of the dilator. Guidewire lumen 103 may be sized to accept a guidewire of a predetermined size. For example, guidewire lumen 103 may be sized to accept a 0.018″ or 0.035″ guidewire. FIG. 8 illustrates the use of a dilator 100 with a guidewire 150.

In use, an incision may be made, and entrance into a desired location (e.g., needle puncture) obtained for a guidewire 150 to be inserted through the incision/needle and advanced to the desired target location. Dilator 110 may be advanced over guidewire 150, the conical tip 105 of dilator 110 slowly opening the passageway (e.g., tissue) until the dilator's first segment 110 is reached, and the passageway is sized according to the first segment (e.g., 5 French) so that the dilator may be removed and another instrument (e.g., catheter, balloon, stent, ablation element, etc.) advanced over the guidewire. If a differently-sized passageway is desired, the user may continue to advance the dilator instead of removing it so that first taper 115 gradually opens the passageway until the size of the passageway is enlarged to a second size corresponding to the second segment 120. This process of advancing the dilator through successive tapered portions and to cylindrical segments of different sizes may be continued until the proper size is achieved. Because the cylindrical segments are of a known diameter, the proper sized passageway may be formed with precision.

Thus, a single dilator 100 may be used to replace an array of dilators of varying sizes. The specific and known gradations of dilator 100 may allow for dilatation of multiple specific and known French sizes by one device, as required by its operator. By using the tapered portions, dilator 100 may also allow the physician to incrementally increase the size of the tract (perhaps from 7 French to 7.5 French) just enough to allow for safe passage of the device without requiring the placement of an even larger dilator or sheath, which is a benefit to both the patient and the operator.

To further increase the usability of the device, an identification system may be utilized so that the cylindrical segments of the dilator and their corresponding sizes or outer diameters may be quickly recognized by the operator. For example, FIG. 9 is a schematic illustration of a dilator with colored segments. In this example, segments 110, 120, 130, 140 are color-coded with colors c1,c2,c3,c4 so that first segment 110 includes a first color (e.g., gray), second segment 120 includes a second color (e.g., green), third segment 130 includes a third color (e.g., orange), and fourth segment 140 includes a fourth color (e.g., blue). In this example, the user may quickly identify the size of the segment by recognizing the color code on the segment to know the size the tract or passageway is enlarged to. It will be understood that the coloring may be added to the entire cylindrical segments or a portion (e.g., a majority) of the outer surface of the cylindrical segments. Additionally, shading, and/or patterns may be used, in addition to or, instead of, the color code to aid operators who may be colorblind. Numerical markings n1,n2,n3,n4 may also be used to identify the size of the cylindrical segments as shown in FIG. 10. In this example, a number is disposed on each cylindrical segment, the number corresponding to the French size. It will be understood that each segment may include a plurality of numerical codes (e.g., two, three of four codes) disposed about the circumference of the cylindrical segment so that the numerical code can be clearly observed without having to rotate the dilator. A plurality of shaded or color-coded annular rings r1,r2,r3,r4 or markings may also be used to identify the cylindrical segments and each segment may include one or more of the annular rings to identify its diameter (FIG. 11).

Thus, a graded access dilator may be used to replace an array of conventional devices and improve an operator's ability to successfully perform a desired intervention. Additionally, the graded access dilators of the instant disclosure will improve patient safety by reducing the risks associated with multiple dilator exchanges as well as reduce the cost and workload for facilities currently having to stock multiple conventional dilator sizes.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with others of the described embodiments.

What is claimed is:

1. A tapered dilator comprising:
a plurality of cylindrical segments having a number of different diameters;
a plurality of tapered portions, each of the plurality of tapered portions being disposed between adjacent ones of the plurality of cylindrical segments and serving as a transition between the adjacent ones of the plurality of cylindrical segments; and
a plurality of markings disposed, each of the plurality of marking being disposed on an outer surface of each of the plurality of cylindrical segments.

2. The tapered dilator of claim 1, wherein the plurality of cylindrical segments include multiple diameters, the plurality of cylindrical segments being arranged such that the multiple diameters increase from a distal end to a proximal end.

3. The tapered dilator of claim 1, wherein a first of the plurality of cylindrical segments is 5 French.

4. The tapered dilator of claim 3, wherein a second of the plurality of cylindrical segments is 6 French.

5. The tapered dilator of claim 4, wherein a third of the plurality of cylindrical segments is 7 French.

6. The tapered dilator of claim 5, wherein a fourth of the plurality of cylindrical segments is 8 French.

7. The tapered dilator of claim 5, wherein each of the plurality of tapered portions are conical and include a first diameter adjacent a first end and a second diameter adjacent a second end, the first diameter and the second diameter having a difference of one French.

8. The tapered dilator of claim 1, wherein the plurality of markings include a designated color for each of the plurality of cylindrical segments.

9. The tapered dilator of claim 1, wherein each designated color is disposed on at least a portion of a respective cylindrical segment.

10. The tapered dilator of claim 1, wherein the plurality of markings include an annular marking on each of the plurality of cylindrical segments.

11. The tapered dilator of claim 10, wherein the annular marking corresponds to a known diameter.

12. The tapered dilator of claim 1, further comprising a guidewire lumen extending through each of the plurality of cylindrical segments and each of the plurality of tapered portions.

13. A tapered dilator comprising:
a plurality of cylindrical segments having a number of different diameters, each of the plurality of cylindrical segments having a distinct color;
a plurality of tapered portions, each of the plurality of tapered portions being disposed between adjacent ones of the plurality of cylindrical segments and serving as a transition between the adjacent ones of the plurality of cylindrical segments; and
wherein the plurality of cylindrical segments and the plurality of tapered portions define a continuous guidewire lumen extending from a proximal end to a distal end.

14. The tapered dilator of claim 13, wherein the plurality of cylindrical segments include multiple diameters, the plurality of cylindrical segments being arranged such that the multiple diameters increase from a distal end to a proximal end.

15. The tapered dilator of claim 13, wherein a first of the plurality of cylindrical segments is 5 French, a second of the plurality of cylindrical segments is 6 French, a third of the plurality of cylindrical segments is 7 French, and a fourth of the plurality of cylindrical segments is 8 French.

16. The tapered dilator of claim 13, wherein each of the plurality of tapered portions are conical and include a first diameter adjacent a first end and a second diameter adjacent a second end, the first diameter and the second diameter having a difference of one French.

17. The tapered dilator of claim 13, further comprising at least one annular ring on each of the plurality of cylindrical segments.

18. The tapered dilator of claim 17, wherein the at least one annular ring corresponds to a known diameter.
